(19) United States
(12) Patent Application Publication
(10) Pub. No.: US 2013/0172734 A1
(43) Pub. Date: Jul. 4, 2013

(54) FLOW MEASUREMENT WITH TIME-RESOLVED DATA

(71) Applicant: Jiang Hsieh, Brookfield, WI (US)

(72) Inventor: Jiang Hsieh, Brookfield, WI (US)

(73) Assignee: General Electric Company, Schenectady, NY (US)

(21) Appl. No.: 13/341,164

(22) Filed: Dec. 30, 2011

(51) Int. Cl. A61B 5/026; A61B 5/021; A61B 6/03; A61B 6/00

(52) U.S. Cl. 600/425; 600/431

(57) ABSTRACT

Estimation of blood flow parameters using non-invasive imaging techniques is described. In one implementation, temporally distinct image volumes are generated. Each respective image volume depicts a respective spatial distribution of a contrast agent within an imaged volume at a different time. The contrast agent movement at each different time from the plurality of image volumes is used in the estimation of a parameter related to the flow of blood within the imaged volume.

### BACKGROUND

Non-invasive imaging technologies allow images of the internal structures or features of a patient to be obtained without performing an invasive procedure on the patient. In particular, such non-invasive imaging technologies rely on various physical principles, such as the differential transmission of X-rays through the target volume, to acquire data and to construct images or otherwise represent the observed internal features of the patient.

One application that may benefit from the use of non-invasive technologies is the determination of fractional flow reserve (FFR). FFR is a technique used to measure pressure differences across a partial blockage (e.g., a coronary artery stenosis) to determine the likelihood that the blockage impedes oxygen delivery to the heart muscle. Conventionally, FFR is an invasive procedure involving insertion of a catheter into the coronary vasculature.

However, conventional applications of non-invasive imaging to the determination of FFR require extensive computational resources. Further, the success of such non-invasive imaging approaches to FFR determination may be limited due to inaccuracies related to the spatial resolution of the imaging modality and/or due to motion artifacts present in the generated images.

### BRIEF DESCRIPTION

In one embodiment, a method for estimating blood flow is provided. The method comprises reconstructing a plurality of temporally distinct image volumes. Each respective image volume depicts a respective spatial distribution of a contrast agent within an imaged volume at a different time. Measures of the contrast agent movement at each different time are derived from the plurality of image volumes. A parameter related to the flow of blood within the imaged volume is estimated based on the derived measures of contrast movement at each different time. In one implementation, the principles of fluid dynamics maybe utilized to derive the pressure differential parameters from the geometry of the lumen and the blood flow parameter.

In accordance with a further embodiment, an imaging system is provided. The imaging system comprises an X-ray source and detector configured to rotate about an imaging volume and to collect projection data over a time interval. The imaging system also comprises one or more processing components configured to receive the projection data and to execute one or more routines. The routines, when executed, cause acts to be performed comprising: reconstructing the projection data to generate a plurality of temporally distinct image volumes, each respective image volume depicting a respective spatial distribution of a contrast agent within the imaging volume at a different time; deriving measures of the contrast agent movement at each different time from the plurality of image volumes; and outputting a parameter related to the flow of blood within the imaging volume based on the derived measures of contrast movement at each different time. In one implementation, the anatomic information (e.g. the size and shape of the lumen) is combined with the blood flow parameter to derive an estimation of the pressure difference or distribution along the blood vessel.

In accordance with an additional embodiment, one or more non-transitory computer-readable media encoding one or more routines are provided. The one or more encoded routines, when executed on a processor, cause act to be performed comprising: generating a plurality of temporally distinct image volumes, each respective image volume depicting a respective spatial distribution of a contrast agent within an imaged volume at a different time; deriving measures of the contrast agent movement at each different time from the plurality of image volumes; and outputting a parameter related to the flow of blood within the imaged volume based on the derived measures of contrast movement at each different time.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a block diagram depicting components of a computed tomography (CT) imaging system, in accordance with aspect of the present disclosure;

FIG. 2 depicts an idealized contrast arrival intensity curve, in accordance with aspect of the present disclosure;

FIG. 3 depicts a contrast arrival intensity curve exhibiting a gradual transition, in accordance with aspect of the present disclosure;

FIG. 4 is a flow chart of an embodiment of a method of flow estimation, in accordance with aspect of the present disclosure;

FIG. 5 depicts a spatial contrast density curve, in accordance with aspect of the present disclosure;

FIG. 6 depicts a temporal contrast density curve, in accordance with aspect of the present disclosure;

FIG. 7 depicts two time-density curves collected at different spatial locations, in accordance with aspect of the present disclosure;

FIG. 8 depicts two spatial density curves collected at different spatial locations, in accordance with aspect of the present disclosure; and

FIG. 9 depicts an example of a non-constant diameter vessel, in accordance with aspect of the present disclosure.

### DETAILED DESCRIPTION

As discussed herein, fractional flow reserve is determined using a flow measurement approach derived using time-resolved computed tomography angiography (CTA) or other suitable modalities. In one such implementation, flow measurements made using time-resolved CTA can be used to derive flow measurements, such as flow velocity, for the heart and coronary vasculature. However, it should also be appreciated that flow measurements may also be derived for non-cardiac applications, such as for the vasculature related to the brain, liver, or other organs. Further, accurate measurement of blood flow at different locations inside a vessel, as discussed herein, allows the estimation of other physical parameters, such as pressure, within the vessel.

It should be also noted that, although time-resolved CTA is primarily described throughout the present discussion, this modality is discussed by way of example only and other methodologies can also be used. Indeed, to the extent that one example or embodiment, such as CTA, is described in a particular context, such discussion is made merely to facilitate explanation by providing a particular context and specific example. However, such explanations are not intended to exclude or preclude the use of other approaches or modalities that provide the same or similar suitable vascular data.

For example, CTA allows generation of cross-sectional information of a vessel by removing overlapping structures in the human body. For some clinical applications, such as the determination of blood flow in carotid arteries, other anatomies do not impact the determination of the contrast density measurement directly from the projections. That is, in such applications, non-CTA modalities may also be useful in the estimation of blood flow. For example, simple X-ray radiography (such as in fluoro mode) can be used to obtain blood velocity calculations. Alternatively, a CT scanner can be used to generate such projection measurements by parking the tube-detector at the desired orientation and obtaining successive set of projections. In other cases, digital subtraction angiography (DSA) can be used to remove other high-density anatomies which do not have iodine contrast uptake. For example, in the imaging of extremities, the arm and leg bones can obstruct the measurement of the iodine contrast in the vessel. Since little motion is present during the imaging of such organ, difference projections (images) between the first measurement (prior to iodine contrast uptake) and the subsequent measurements (post-iodine contrast) may be obtained to show the “vessel” only projections (images). These projections (images) can be used to estimate the blood velocity using the same approach discussed herein. One benefit of non-CTA approaches is the reduced dose to patient, since only limited numbers of projections may be collected over time to arrive at the desired measurements.

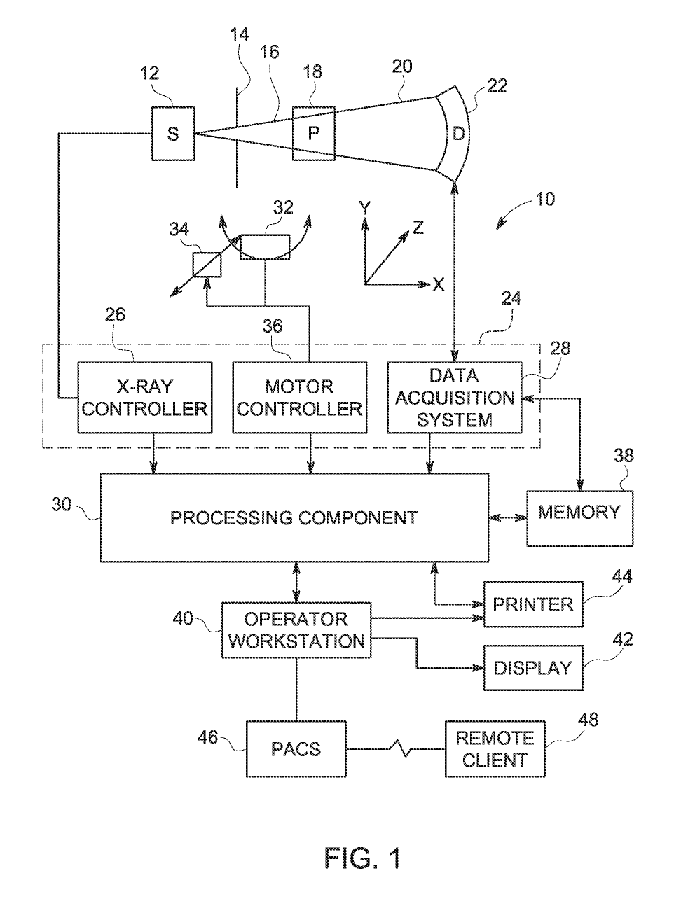

However, to the extent that CTA is a useful modality for explaining the concepts discussed herein, it may be useful to provide a brief description of basic components of a CT system that may be used in accordance with the present disclosure. For example, turning to FIG. 1, a CT imaging system 10, such as a multi-slice CT system, is depicted that may be used to acquire X-ray attenuation data at a variety of view angle positions as the gantry rotates around a patient; these data would be suitable for CTA. In the embodiment illustrated in FIG. 1, imaging system 10 includes a source of X-ray radiation 12 positioned adjacent to a collimator 14. The X-ray source 12 may be an X-ray tube, a distributed X-ray source (such as a solid-state or thermionic X-ray source) or any other source of X-ray radiation suitable for the acquisition of medical or other images.

The collimator 14 permits X-rays 16 to pass into a region in which a patient 18, is positioned. In the depicted example, the X-rays 16 are collimated to a cone-shaped beam and/or a fan-shaped beam that passes through the imaged volume. A portion of the X-ray radiation 20 passes through or around the patient 18 (or other subject of interest) and impacts a detector array, such as a multi-slice detector, represented generally at reference numeral 22. Detector elements of the array produce electrical signals that represent the intensity of the incident X-rays 20. These signals are acquired and processed to reconstruct images of the features within the patient 18.

Source 12 is controlled by a system controller 24, which furnishes both power, and control signals for CTA examination sequences. In the depicted embodiment, the system controller 24 controls the source 12 via an X-ray controller 26 which may be a component of the system controller 24. In such an embodiment, the X-ray controller 26 may be configured to provide power and timing signals to the X-ray source 12.

Moreover, the detector 22 is coupled to the system controller 24, which controls acquisition of the signals generated in the detector 22. In the depicted embodiment, the system controller 24 acquires the signals generated by the detector using a data acquisition system 28. The data acquisition system 28 receives data collected by readout electronics of the detector 22. The data acquisition system 28 may receive sampled analog signals from the detector 22 and convert the data to digital signals for subsequent processing by a processor 30 discussed below. Alternatively, in other embodiments the digital-to-analog conversion may be performed by circuitry provided on the detector 22 itself. The system controller 24 may also execute various signal processing and filtration functions with regard to the acquired image signals, such as for initial adjustment of dynamic ranges, interleaving of digital image data, and so forth.

In the embodiment illustrated in FIG. 1, system controller 24 is coupled to a rotational subsystem 32 and a linear positioning subsystem 34. The rotational subsystem 32 enables the X-ray source 12, collimator 14 and the detector 22 to be rotated one or multiple turns around the patient 18, such as rotated primarily in an x, y-plane about the patient. It should be noted that the rotational subsystem 32 might include a gantry upon which the respective X-ray emission and detection components are disposed. Thus, in such an embodiment, the system controller 24 may be utilized to operate the gantry.

The linear positioning subsystem 34 may enable the patient 18, or more specifically a table supporting the patient, to be displaced within the bore of the CT system 10, such as in the z-direction relative to rotation of the gantry. Thus, the table may be linearly moved (in a continuous or step-wise fashion) within the gantry to generate images of particular areas of the patient 18. In the depicted embodiment, the system controller 24 controls the movement of the rotational subsystem 32 and/or the linear positioning subsystem 34 via a motor controller 36.

In general, system controller 24 commands operation of the imaging system 10 (such as via the operation of the source 12, detector 22, and positioning systems described above) to execute examination protocols (such as CTA protocols) and to process acquired data. For example, the system controller 24, via the systems and controllers noted above, may rotate a gantry supporting the source 12 and detector 22 about a subject of interest so that X-ray attenuation data may be obtained at a variety of view angle positions relative to the subject. In the present context, system controller 24 may also include signal processing circuitry, associated memory circuitry for storing programs and routines executed by the computer (such as routines for executing image processing or analysis techniques described herein), as well as configuration parameters, image data, and so forth.

In the depicted embodiment, the image signals acquired and processed by the system controller 24 are provided to a processing component 30 for measurement data processing and/or reconstruction of images. The processing component 30 may be one or more conventional microprocessors. The data collected by the data acquisition system 28 may be transmitted to the processing component 30 directly or after storage in a memory 38. Any type of memory suitable for storing data might be utilized by such an exemplary system 10. For example, the memory 38 may include one or more optical, magnetic, and/or solid state memory storage structures. Moreover, the memory 38 may be located at the acquisition system site and/or may include remote storage devices for storing data, processing parameters, and/or routines for image reconstruction, as described below.

The processing component 30 may be configured to receive commands and scanning parameters from an operator via an operator workstation 40, typically equipped with a keyboard and/or other input devices. An operator may control the system 10 via the operator workstation 40. Thus, the operator may observe the reconstructed images and/or otherwise operate the system 10 using the operator workstation 40. For example, a display 42 coupled to the operator workstation 40 may be utilized to observe the reconstructed images and to control imaging. Additionally, the images may also be printed by a printer 44 which may be coupled to the operator workstation 40.

Further, the processing component 30 and operator workstation 40 may be coupled to other output devices, which may include standard or special purpose computer monitors and associated processing circuitry. One or more operator workstations 40 may be further linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, virtual private networks, and so forth.

It should be further noted that the operator workstation 40 may also be coupled to a picture archiving and communications system (PACS) 46. PACS 46 may in turn be coupled to a remote client 48, radiology department information system (RIS), hospital information system (HIS) or to an internal or external network, so that others at different locations may gain access to the raw or processed image data.

While the preceding discussion has treated the various exemplary components of the CT imaging system 10 separately, these various components may be provided within a common platform or in interconnected platforms. For example, the processing component 30, memory 38, and operator workstation 40 may be provided collectively as a general or special purpose computer or workstation configured to operate in accordance with the aspects of the present disclosure. In such embodiments, the general- or special-purpose computer may be provided as a separate component with respect to the data acquisition components of the system 10 or may be provided in a common platform with such components. Likewise, the system controller 24 may be provided as part of such a computer or workstation or as part of a separate system dedicated to image acquisition. In a present embodiment, the CT imaging system 10 may be a system suitable for coronary CT angiography (CCTA) or other imaging applications suitable for imaging of the vasculature. For example, a suitable CT imaging system may be a multi-slice CT scanner (e.g., 4-slice, 16-slice, 64-slice and so forth) or a cone-beam CT scanner. The CT scanner may have a rotation speed between about 0.35 seconds to about 0.5 seconds for a full gantry rotation.

As may be appreciated, imaging of the vasculature using X-ray based techniques (such as CTA) typically employs a contrast agent (such as an iodine-based agent) that is administered to the patient to temporarily increase X-ray opacity of the blood vessels undergoing imaging. When the coverage of detector 22 covers a substantial fraction of an organ, the contrast agent can be dynamically monitored via an imaging modality, such as CTA, as it flows through the vessels.

Due to the flow of the blood within a vessel and the dissipation of the contrast agent over time, the intensity of the iodine contrast inside a vessel is not constant over time. Often, a gradient can be observed in the contrast spatial distribution. Further, when imaging an organ during its contrast uptake (or washout) phase, the progression of the contrast flow can be observed in the generated images. In accordance with embodiments of the present approach and as discussed below, these observations may be leveraged to allow the estimation of the blood flow inside a vessel.

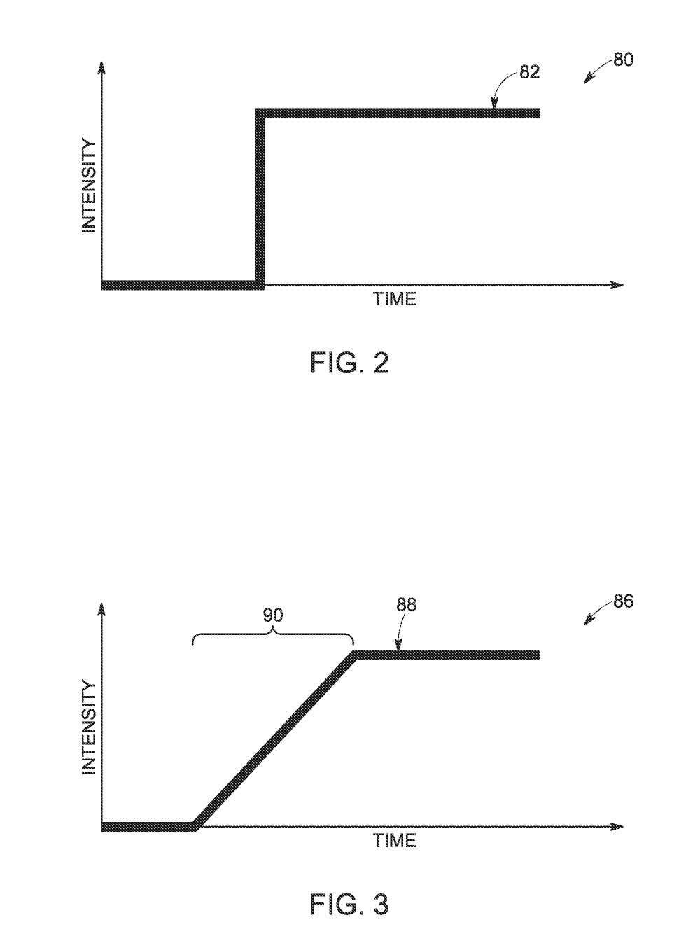

For example, turning to FIGS. 2 and 3, FIG. 2 depicts an idealized contrast arrival intensity curve 80 where contrast arrival at a spatial location is characterized by a clean step function 82. That is, in the idealized scenario, there is no contribution to intensity by the contrast agent until the instant when the contrast agent arrives at the location in question, at which point the increase in intensity is instantaneous and is at its maximum.

In practice, however, the contrast arrival intensity curve 86 may be characterized by a gradual transition (FIG. 3) that may be linear or non-linear in nature. for instance, in the depicted example, of FIG. 3, the contrast arrival intensity 88 may be characterized as a gradual transition that is substantially linear over a period of time 90 corresponding to the increase in contrast at the site (i.e., the contrast-rise phase). Therefore, a simple threshold to detect the arrival of contrast at a site may not be reliable, especially in the presence of noise.

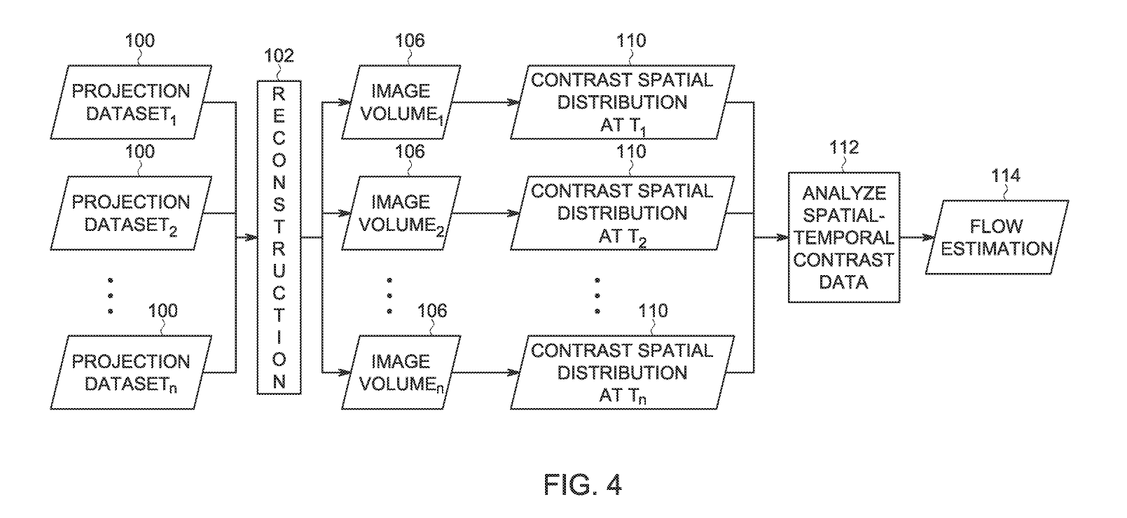

With this in mind, in accordance with one or more embodiments the coverage of the detector 22 in the z-direction (i.e., along the axis about which the source 12 and detector 22 rotate) is leveraged to more accurately estimate blood flow. In particular, the reconstructed volumes over different ranges of projections provide dynamic information about the flow of contrast over time. Thus, the combination of the spatial-temporal information derived from the reconstructed volumes can be used to accurately estimate the flow information. An embodiment of one such process is graphically represented in FIG. 4 where respective sets of projection data 100, such as may be acquired in accordance with a CTA scan protocol, are reconstructed (block 102) to generate respective image volumes 106 that are temporally distinct from one another (i.e., graphically depict the volume or vasculature of interest at different times). From these temporally distinct image volumes 106, the contrast spatial distribution 110 at each time of interest may be determined. The spatial and temporal information represented in these temporally distinct contrast spatial distributions 110 may in turn be analyzed (block 112), as discussed herein, to generate an estimate 114 of the flow of blood within the volume or vasculature of interest. Further, it should be noted that the contrast spatial distribution discussed above is not limited to particular orientations, such as along the z-axis. For example, the spatial distribution of the contrast can be determined along the lumen of a curved vessel, or along multiple branches of a vessel before and after the bifurcation. The spatial distribution of contrast can be determined along the centerline of a vessel (or its lumen), or it can be the integrated intensities over the cross-section of the lumen.

With regard to the modeling that may be employed to generate such estimates in accordance with this approach, in one basic example the four-dimensional contrast density distribution may be denoted as f(r,t), where r is a three-dimensional vector in space and t is a variable over time. Thus, f(r,t), describes the spatial density distribution at a particular time, t₀, and f(r₀,t) denotes the time density curve at a particular vessel location, r₀. If r₀ and r₁ are denoted as two nearby locations along a single vessel (without bifurcation or stenosis) the following can be assumed:

f(r₀,t)≈f(r₁,t+Δt) (1).

That is, the contrast density curve, f(r₁,t), at a location r₁ slightly downstream from the location r₀ is simply a time delayed density curve of f(rₒ,t). This assumption can be justified based on the conservation of iodine contrast and blood (no blood or contrast lose between the two locations due to lack of bifurcation), and the close proximity of the two locations so the dilution of contrast can be assumed to be negligible. After the tomographic reconstruction process, the contrast density curve of the reconstructed image becomes q(r₁,t), and can be approximated by the integration of the function ƒ(r₁,t) over a time window Γ. Equality described by equation (1) still holds:

q(r₀,t)=∫₀^{Γ}w(t′)f(r₀,t−t′)dt≈q(r₁,t+Δt)=∫₀^{Γ}w(t′)f(r₁,t+Δt−t′)dt (2)

where w(t) accounts for the weighting function, filter kernels, and interpolation functions used in the tomographic reconstruction process. Assuming the flow rate does not change between r₀ and r₁, this simplifies to:

$$q ( r , {t}_{0} ) = q ( {r}_{1} , \frac{r - {r}_{1}}{v} ) , where {r}_{0} \leq r \leq {r}_{1} \qquad ( 3 )$$

where v is the blood flow velocity (i.e., the distance traveled by a blood element is simply the product of velocity and time). As indicated by equation (3), the contrast density curve over space between r₀ and r₁ has the same shape as the scaled time density curve (by stretching or compressing the x-axis) measured over a time period that allows the blood to flow from r₀ to r₁. Therefore, by matching the two curves over time (e.g., using the minimum least square fit), the blood flow velocity, v, can be reliably calculated since the distance r₁-r₀ is known.

A simulation was performed to test the preceding approach. In this simulation a vertical tube was simulated (for simplicity of analysis and calculation) having a radius of 3 mm and was filled with blood and iodine mixture with a linear gradient of 20 HU/s over time and reaches the peak of 300 HU. The blood flowed at a velocity of 130 mm/s. The CT acquisition speed was 0.35 s per rotation with 984 views/rotation, and covered 160 mm over z (i.e., along the axis of rotation of the CT system). A set of projections were simulated over five gantry rotations with and without noise, and half-scan reconstruction was carried out to generate two sets of four-dimensional images (with and without noise).

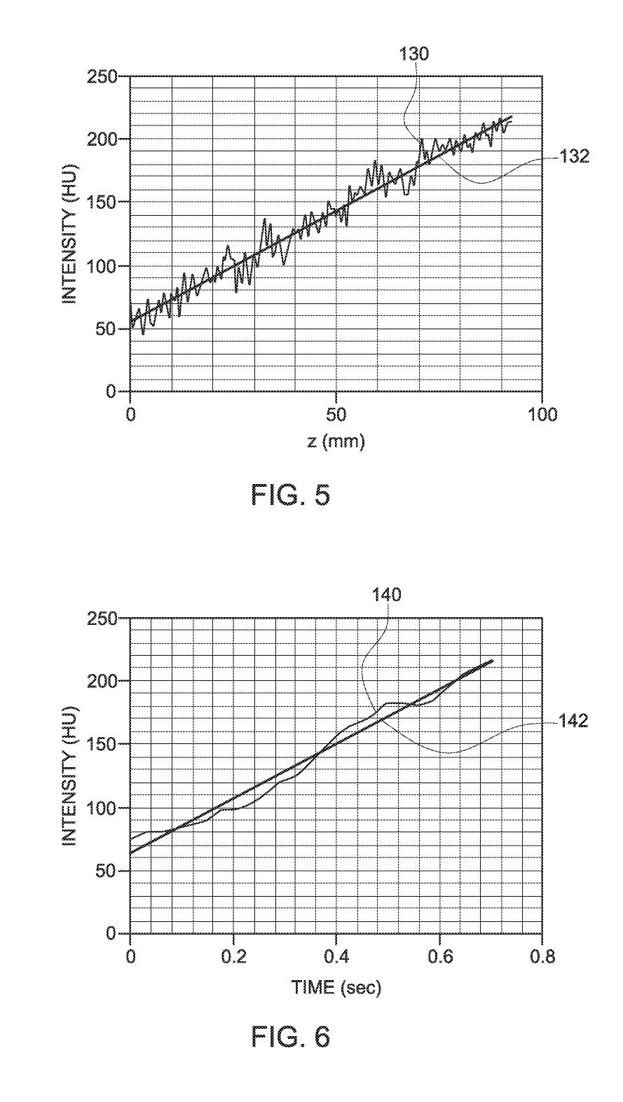

Based on the simulated data, the spatial (i.e., distance) and temporal contrast density curves are plotted in FIGS. 5 and 6, respectively. In particular, FIG. 5 depicts a spatial contrast density curve 130 corresponding to the intensity observed in the noisy image while spatial contrast density curve 132 corresponds to the intensity observed in the noise-free image. Similarly, in FIG. 6, a temporal contrast density curve 140 corresponding to the intensity observed in the noisy image is depicted in addition to a temporal contrast density curve 142 corresponding to the intensity observed in the noise-free image. To demonstrate equation (3) above with respect to the depicted plots, the z-coverage of FIG. 5 (i.e., 91 mm) is equal to the time span of FIG. 6 (i.e., 0.7 s) multiplied by the velocity (130 mm/s).

Further, when the horizontal axis is properly scaled, the paired corresponding curves match in terms of slope and shape. That is, by scaling the horizontal axis of the time density curve (FIG. 6), a match is obtained, in a minimum least square error sense, between the spatial density curve (FIG. 5) and the scaled temporal density curve. The scaling factor for the horizontal axis is then the blood velocity, v. Thus, equation (3) appears to provide an accurate method to calculate blood flow.

If it is assumed that over a short distance and over a short time period the density curves are substantially linear, the velocity may be estimated. For example, a linear fit of the spatial density curve vs. distance may be performed to obtain a DC and linear coefficient, c_{z}(0) and c_{z}(1). Similarly, a linear fit of the temporal density curve vs. time may be performed to obtain a DC and linear coefficient, cₜ(0) and cₜ(1). The following formula can then be used to calculate the velocity:

$$v = \frac{{c}_{t} ( 1 )}{{c}_{z} ( 1 )} \qquad ( 4 )$$

Table 1 shows the calculated results based on equation (4) for the noise-less and noisy cases described above. The standard deviation of the reconstructed noisy images is roughly 20 HU, which is similar to many clinical cardiac images. The accuracy of the estimated blood flow is good (i.e., the simulated blood flow was 130 mm/s)

TABLE 1

| | | | | | Flow |
|---|---|---|---|---|---|
| | c_{z} (0) | c_{z} (1) | cₜ (0) | cₜ (1) | (mm/s) |
| Noise-less | 54.89 | 1.77 | 54.96 | 229.57 | 129.99 |
| Noisy | 56.62 | 1.74 | 62.83 | 215.45 | 123.56 |
| (s = 20.2 HU) | | | | | |

While the preceding discussion relates to one approach for estimating blood flow, in other implementations other assumption or considerations may hold. For example, in one implementation only coarse samples along z are available, such as the case of organ perfusion. In one such embodiment, thick slices (such as 5 mm) of image data are acquired over a small z-coverage (e.g., 20 mm or 40 mm) while images are reconstructed at fine time intervals. In such an embodiment, certain of the assumption discussed above may not apply.

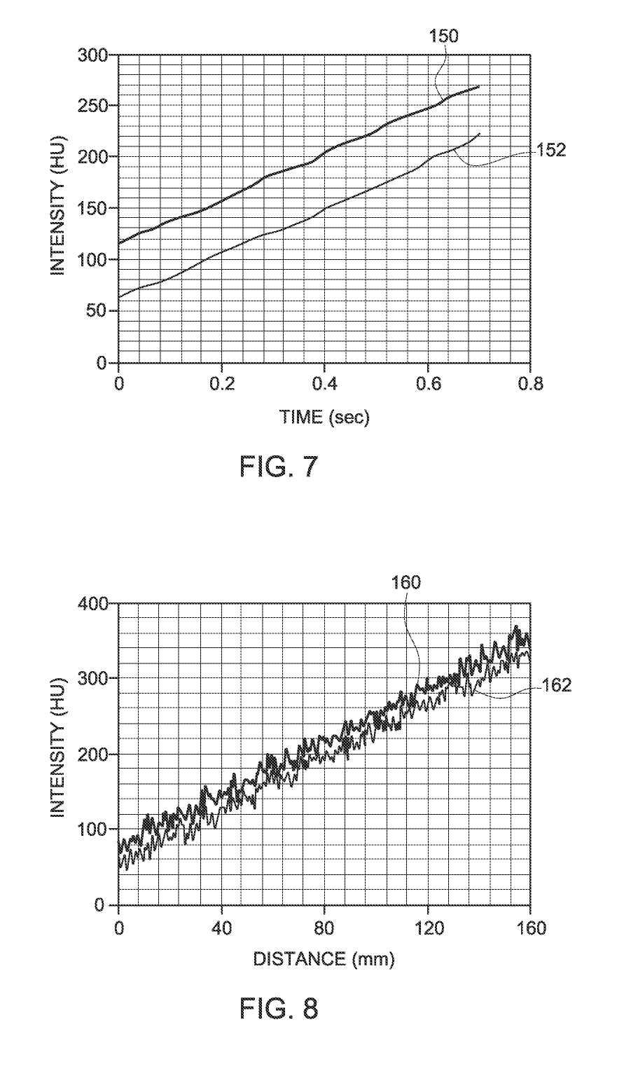

In such an implementation, the assumption described above with respect to equation (1) (namely, that the contrast time density curve at a downstream location r₁ is simply a delayed contrast density curve at location r₀) may be revisited to address this scenario. In particular, if the time density curves at two locations are plotted, one should be a simple shift of the other. For example, turning to FIG. 7, two time density curves (curves 150 and 152) are shown that are 30 mm apart. By estimating the amount of the shift, Δt, such that the two curves overlap, the blood flow is then simply:

$$v = \frac{D}{Δ t} \qquad ( 5 )$$

where D is the distance between the two sampling locations. If the curves are assumed to be linear over the short time span, both curves can be fitted to obtain the DC and linear coefficients for: c_{t,r0}(0), c_{t,r0}(1), c_{t,r1}(0), and c_{t,r1}(t). The velocity can then be calculated as:

$$v = \frac{D ⌊ {c}_{t , r 1} ( 1 ) + {c}_{t , r 0} ( 1 ) ⌋}{2 [ {c}_{t , r 1} ( 0 ) - {c}_{t , r 0} ( 0 ) ]} \qquad ( 6 )$$

It may be noted that results derived using equation (6) may be sensitive to the spacing between the samples.

Although the preceding approaches are effective in calculating the velocity of the blood flow, these approaches typically utilize the scan of an organ over an extended period of time to generate the time-density curves. Such an extended scan may be unavailable or undesired in certain contexts, such as where dose to which the patient is exposed is to be limited.

To address this issue, an approach may be derived that utilizes minimal additional data over time. By way of example, consider two spatial density curves taken 88 ms apart (FIG. 8, curves 160 and 162). In this acquisition, the original half-scan acquisition is only extended an extra 88 ms, less than 40% increase in dose as compared to a conventional minimum data acquisition for cardiac. For neural application, this is only a 25% increase in dose compared to a conventional minimum data acquisition. As in preceding examples, one curve is a simple shift of another. If it is assumed that the contrast density curve is linear over a short distance, the DC and linear coefficients may be obtained for the two curves: c_{r,t0}(0), c_{r,t0}(1), c_{r,t1}(0), and c_{r,t1}(t). The velocity can be calculated as:

$$v = \frac{2 ⌊ {c}_{r , t 1} ( 0 ) - {c}_{r , t 0} ( 0 ) ⌋}{Δ t ⌊ {c}_{r , t 1} ( 1 ) + {c}_{r , t 0} ( 1 ) ⌋} \qquad ( 7 )$$

where Δt is the time difference between the two density curves. By way of comparison, the performance of the different approaches (on simulated noisy and noise-free data having flow rate of 130.00 mm/s) described above is provided in Table 2.

TABLE 2

| | Equation (4) | Equation (6) | Equation (7) |
|---|---|---|---|
| Noise-Free | 129.99 | 129.90 | 130.34 |
| Noisy | 123.56 | 123.76 | 132.93 |

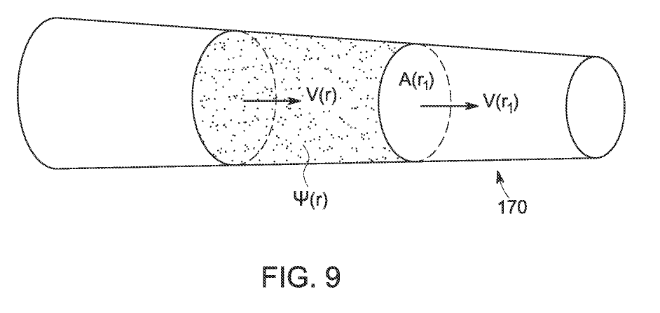

The examples discussed above assume a constant vessel diameter. For vessels that change in size, the flow rate is inversely proportional to the cross section area based on the conservation of blood. Therefore, additional scaling may be needed to take into consideration of the vessel diameter change. To account for the vessel size change, the property of the conservation of blood-contrast volume may again be relied upon. If ψ(r) is denoted as the total fluid volume between location r and r₁ as shown in FIG. 9 (depicting a vessel 170 of non-constant diameter), this value may be expressed as:

ψ(r)=∫ᵣ₁^{r}A(r)dr (8)

The rate of discharge at location r₁ is the product of the cross-sectional area, A(r₁), and the velocity, v(r₁). The time, t, it takes for the fluid at location r to pass through r₁ is simply the time to pass the entire volume u(r):

$$t = \frac{Ψ ( r )}{A ( {r}_{1} ) v ( {r}_{1} )} \qquad ( 9 )$$

Incorporating this expression of t into equation (3) yields:

$$q ( r , {t}_{0} ) = q [ {r}_{1} , \frac{Ψ ( r )}{A ( {r}_{1} ) v ( {r}_{1} )} ] , where {r}_{0} \leq r \leq {r}_{1} \qquad ( 10 )$$

Note that in equation (10), the quantities ψ(r) and A(r₁) can be measured directly from CTA images. The quantity, ψ(r)/A(r₁), is the “equivalent distance” between r and r₁ that holds the same blood volume if the cross-section of the vessel were constant. With this interpretation, the similarity between equations (3) and (10) may be noted. Equation (10) states that the spatial density curve, q(r, t₀), at a particular time instant, t₀, is a nonlinearly scaled (along the horizontal axis) time density curve, q(r₁, t), at a particular downstream location, r₁. The scaling factor is the velocity v(r₁) at the location r₁. Similar to the constant diameter vessel case, by fitting the measured spatial density curve and time density curve, we obtain the blood velocity.

In the same manner, we arrive at the counterpart of equation (2) for a variable size vessel:

$$q ( {r}_{0} , t ) = q [ {r}_{1} , t + Δ t ] , where Δ t = \frac{Ψ ( {r}_{0} )}{A ( {r}_{1} ) v ( {r}_{1} )} \qquad ( 11 )$$

where ψ(r₀) is the vessel volume between r₀ and r₁. This equation states that two time density curves measured at two different locations along a vessel have the same shape and are shifted (along the time axis) relative to one another. Based on equations (10) and (11), the blood flow velocities can be estimated for the various approaches outlined above in the context of a vessel of non-constant diameter.

While the preceding describes various approaches for measuring blood flow velocity at different points within a vessel, it should be appreciated that such measures may in turn be used to derive other parameters of interest such as a fractional flow reserve or an intra-vessel pressure. To derive such parameters, both the anatomical information (size and shape of the lumen) and flow information can be combined. In the derivation of such parameters, fluid dynamic principles (e.g. Bernoulli's principle), can be used. In particular, difference in blood flow velocities on the respective upstream and downstream sides of an obstruction, such as a stenosis, may be useful in evaluating the effect of the obstruction on blood flow and/or in making a diagnosis related to a patient's cardiovascular health.

Technical effects of the invention include the estimation of blood flow parameters using non-invasive imaging techniques. For example, blood flow velocity and/or fractional flow reserve may be non-invasively assessed. In one embodiment, blood flow measurement for organs (such as the heart or brain) or associated vasculature may be obtained using time-resolved CTA. In one embodiment, time-resolved CTA is used to estimate fractional flow reserve.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for estimating blood flow, comprising:
obtaining a plurality of temporally distinct image, wherein each respective image depicts a respective spatial distribution of a contrast agent within an imaged vessel at a different time; and
estimating a parameter related to the flow of blood within the imaged vessel based on the temporal distribution and spatial distribution of the contrast agent.

2. The method of claim 1, wherein the parameter comprises a blood flow velocity or a pressure.

3. The method of claim 1, wherein the parameter comprises a fractional flow reserve.

4. The method of claim 1, wherein estimating the parameter comprises deriving one or more combined spatial and temporal contrast density functions from the plurality of images.

5. The method of claim 1, wherein estimating the parameter comprises estimating velocity by performing a linear fit of a spatial density curve versus distance and performing a linear fit of a temporal density curve versus time.

6. The method of claim 1, wherein estimating the parameter comprises estimating velocity by determining a ratio of a first linear coefficient relative to a second linear coefficient, wherein the first linear coefficient is related to the fit of a temporal density curve versus time and the second linear coefficient is related to the fit of a spatial density curve versus distance.

7. The method of claim 1, wherein the plurality of temporally distinct images comprise reconstructed CT image volumes, projection measurements, or digital subtraction angiograms.

8. An imaging system, comprising:
an X-ray source and detector configured to cooperatively image a field of view over a time interval;
one or more processing components configured to receive the projection data and to execute one or more routines, wherein the routines, when executed, cause acts to be performed comprising:
reconstructing one or more signals generated by the detector to generate a plurality of temporally distinct images, each respective image depicting, at a different time, a spatial distribution of a contrast agent within a vessel within the field of view; and
estimating a parameter related to the flow of blood within the vessel based on the temporal distribution and spatial distribution of the contrast agent.

9. The imaging system of claim 8, wherein the imaging system comprises one of a computed tomography angiography system, an X-ray radiography system, a computed tomography system with a fixed gantry location, or a digital subtraction angiography system.

10. The imaging system of claim 8, wherein the parameter comprises a blood flow velocity or a pressure.

11. The imaging system of claim 8, wherein the parameter comprises a fractional flow reserve.

12. The imaging system of claim 8, wherein the one or more processing components estimate the parameter by deriving one or more combined spatial and temporal contrast density functions from the plurality of images.

13. The imaging system of claim 8, wherein the parameter comprises a velocity and wherein the one or more processing components derive the velocity by performing a linear fit of a spatial density curve versus distance and performing a linear fit of a temporal density curve versus time.

14. The imaging system of claim 8, wherein the parameter comprises a velocity and wherein the one or more processing components estimate the velocity by determining a ratio of a first linear coefficient relative to a second linear coefficient, wherein the first linear coefficient is related to the fit of a temporal density curve versus time and the second linear coefficient is related to the fit of a spatial density curve versus distance.

15. One or more non-transitory computer-readable media encoding one or more routines, wherein the one or more encoded routines, when executed on a processor, cause act to be performed comprising:
generating a plurality of temporally distinct images, each respective image depicting a respective spatial distribution of a contrast agent within an imaged vessel at a different time; and
estimating a parameter related to the flow of blood within the imaged vessel based on the temporal distribution and spatial distribution of the contrast agent.

16. The one or more computer-readable medial of claim 15, wherein the parameter comprises a blood flow velocity or a pressure.

17. The one or more computer-readable medial of claim 15, wherein the parameter comprises a fractional flow reserve.

18. The one or more computer-readable medial of claim 15, wherein measures of the contrast agent movement at each different time are derived by deriving one or more combined spatial and temporal contrast density functions from the plurality of image volumes.

19. The one or more computer-readable medial of claim 15, wherein the parameter comprises a velocity that is derived by performing a linear fit of a spatial density curve versus distance and performing a linear fit of a temporal density curve versus time.

20. The one or more computer-readable medial of claim 15, wherein the parameter comprises a velocity that is estimated by determining a ratio of a first linear coefficient relative to a second linear coefficient, wherein the first linear coefficient is related to the fit of a temporal density curve versus time and the second linear coefficient is related to the fit of a spatial density curve versus distance.
